(19) United States
(12) Patent Application Publication
(10) Pub. No.: US 2026/0044716 A1
(43) Pub. Date: Feb. 12, 2026

(54) CROSS-MODALITY REPRESENTATION LEARNING

(71) Applicant: DOLBY LABORATORIES LICENSING CORPORATION, San Francisco, CA (US)

(72) Inventors: Eloy Philip Theo GEENJAAR, Atlanta, GA (US); Lie LU, Dublin, CA (US)

(73) Assignee: DOLBY LABORATORIES LICENSING CORPORATION, San Francisco, CA (US)

(21) Appl. No.: 19/291,549

(22) Filed: Aug. 5, 2025

Related U.S. Application Data
(60) Provisional application No. 63/680,988, filed on Aug. 8, 2024.

(51) Int. Cl. G06N 3/0455; G16H 40/67

(52) U.S. Cl. CPC G06N 3/0455; G16H 40/67

(57) ABSTRACT

A computer-implemented method includes processing a time-series input signal using an encoder to produce an encoded representation, segmenting the encoded representation into a plurality of patches, applying a masking operation to a subset of the patches to produce a masked encoded representation, processing the masked encoded representation using a transformer to generate contextual features, processing the contextual features using a decoder to generate a predicted frequency-domain representation of the time-series input signal, and adjusting parameters of the encoder and parameters of the transformer to minimize a loss between the predicted frequency-domain representation and a reference frequency-domain representation derived from the time-series input signal.

### CROSS-REFERENCE TO RELATED APPLICATIONS

This application claims the benefit of U.S. Provisional Application No. 63/680,988 filed Aug. 8, 2024, the entire disclosure of which is incorporated by reference.

### FIELD

The present disclosure relates to machine learning techniques for processing physiological sensor data and, more particularly, to pretraining machine learning models using data from an available domain for inference in a different domain.

### SUMMARY

Machine learning models benefit from high-quality training data to produce reliable and generalizable results at inference time. These models are typically trained to recognize patterns within data, and their effectiveness often depends on the characteristics of the training dataset.

Machine learning training paradigms are commonly categorized as supervised or unsupervised. In supervised learning, the model generally works with labeled data, where each input is paired with a known target output. The goal is typically to learn functional mappings from inputs to outputs that might generalize well to unseen data. Unsupervised learning usually works with unlabeled data without explicit target outputs. Instead, it often aims to uncover intrinsic structures in the data, such as clusters, correlations, or latent representations.

Regardless of paradigm, a model's performance might depend on the availability of large, diverse, high-quality datasets. Both quantity and quality can affect the model's ability to extract meaningful relationships—between input features and, in supervised learning, output labels. Well-curated, representative datasets may enable models to learn generalizable patterns rather than memorizing training data, potentially reducing overfitting risk. Conversely, training on insufficient, noisy, or biased data could produce models that perform well during training but might not generalize effectively to real-world scenarios.

Machine learning offers potential advantages across a wide range of bio-signal analysis applications. These applications may span multiple physiological signal modalities, including, for example, electroencephalography (EEG), electrocardiography (ECG), electromyography (EMG), photoplethysmography (PPG), electrooculography (EOG), and accelerometer-based motion signals.

For instance, machine learning models may be used to analyze EEG signals, which capture brain electrical activity, for applications such as attention detection, seizure classification, sleep stage scoring, or brain-computer interface (BCI) functionality. Similarly, models trained on ECG data, which reflects heart electrical activity, may be used to detect arrhythmias, classify cardiac rhythms (for example, atrial fibrillation), or analyze heart rate variability for diagnostic or biometric purposes.

Models trained on EMG data, which records skeletal muscle electrical activity during contraction, may be used to classify gestures, decode motor intent, help control prosthetic devices, or support neuromuscular disorder diagnosis. Machine learning models may also analyze PPG data, which measures blood volume changes using optical skin sensors, to estimate heart rate, monitor blood oxygen saturation, or detect stress and affective states non-invasively.

Similarly, machine learning models may process accelerometer-based motion signals from human activity recognition (HAR) and gesture recognition applications to possibly classify physical activities, detect postural transitions, or interpret movement patterns in wearable or mobile systems.

While machine learning models have a wide range of applications analyzing various bio-signal modalities, many of these applications are hindered by data scarcity, particularly the lack of available large labeled high-quality datasets for training. Furthermore, data collection in bio-signal domains may be impeded by high costs, patient privacy and ethical concerns, and the need for accurate expert annotation.

However, among bio-signals, EEG data stands out as valuable training data due to its relatively rich availability and well-characterized spectral features. EEG data may be captured via electrodes placed on the scalp, producing complex signals with strong frequency-domain components that are widely studied, well-documented, and broadly available. In contrast, modalities such as EMG, ECG, and PPG (among others) often lack large, diverse, high-quality datasets (especially labeled ones) suitable for training, creating significant technical challenges for training machine learning models in these domains.

While each bio-signal modality captures different physiological processes, many modalities share common characteristics, particularly in the frequency or time-frequency domain. For example, frequency-domain features—such as oscillatory patterns, spectral power distributions, and rhythmic bursts—are prevalent and often similar or generalizable across multiple signal types. These frequency-based characteristics and relationships tend to be modality-agnostic, allowing knowledge based on frequency characteristics to be transferrable across machine learning models for different modalities.

Consequently, a model trained to recognize meaningful frequency structures in a first modality (such as EEG) may effectively apply similar strategies when analyzing data in a second, different modality (such as EMG or PPG), as the data in the second modality often reflects similar patterns in the frequency domain (e.g., underlying physiological rhythms in spectral profiles). Accordingly, by training on spectral or frequency-domain characteristics shared across bio-signal modalities, machine learning models may gain the ability to generalize across different modalities even when training data in the target domain is sparse.

Systems, apparatuses, methods, and techniques described in this specification provide solutions to these and other technical challenges by pretraining a machine learning model using training data in an available domain for inference in a different domain. For example, during pretraining, an encoder (such as a convolutional neural network [CNN]) processes a time-series input signal (e.g., training data in the available domain such as EEG data) to produce an encoded representation. When implemented as a CNN, the encoder extracts local temporal features from the time-series input signal—such as transient waveforms or localized spectral bursts—by applying learnable filters, which are effective in detecting fine-grained local frequency patterns characteristic of physiological signals.

The encoded representation may then be segmented into a series of patches, and a masking operation is applied to a randomly selected subset of these patches. The masked encoded patches may be processed using a transformer network, which leverages temporal self-attention mechanisms to capture global temporal features across time to generate contextual features. The global temporal features captured by the transformer network allow the machine learning model to reason about long-range interactions between signal components which is beneficial for modeling physiological rhythms and state transitions that unfold over extended time windows. The transformer thus complements the CNN by offering a global perspective, enabling the machine learning model to understand both transient and sustained temporal dynamics.

During pretraining, the contextual features output by the transformer network may be passed to a pretraining head—for example, implemented as a decoder—to reconstruct a predicted frequency-domain representation of the original time-series input signal. Parameters of the machine learning model (such as parameters of the encoder and transformer) may be adjusted to minimize reconstruction loss between the predicted frequency-domain representation and a reference frequency-domain representation of the time-series input signal. Because portions of the encoded representation provided as input to the transformer network are masked, the machine learning model is forced to predict missing portions of the signal, encouraging the model to infer broader structural patterns rather than local artifacts.

Furthermore, by training the model to reconstruct masked frequency components, the machine learning model may be encouraged to learn frequency-domain structures and relationships, allowing the pretrained model to learn patterns that are both physiologically relevant and generalizable across different bio-signal domains. For example, many bio-signals—such as EEG, ECG, EMG, PPG—share common frequency-domain or time-frequency properties (e.g., alpha rhythms, heart rate variability, muscle burst frequencies). As a result, the pretrained machine learning model is equipped to generalize to new bio-signal domains with minimal downstream adaptation. Furthermore, since frequency-domain features are typically more stable across subjects and recording conditions, pretraining the machine learning model to reconstruct a frequency-domain representation may improve cross-subject and cross-modal robustness of the model.

According to some examples, a computer-implemented method includes processing a time-series input signal using an encoder to produce an encoded representation, segmenting the encoded representation into a plurality of patches, applying a masking operation to a subset of the patches to produce a masked encoded representation, processing the masked encoded representation using a transformer to generate contextual features, processing the contextual features using a decoder to generate a predicted frequency-domain representation of the time-series input signal, and adjusting parameters of the encoder and parameters of the transformer to minimize a loss between the predicted frequency-domain representation and a reference frequency-domain representation derived from the time-series input signal.

In other features, the masking operation includes masking one or more consecutive sequences of fixed-sized patches from the encoded representation.

In other features, the time-series input signal includes a first modality and a second modality, the method further includes processing the contextual features using a first decoder to generate a first predicted frequency-domain representation of the time-series input signal, processing the contextual features using a second decoder to generate a second predicted frequency-domain representation of the time-series input signal, and adjusting parameters of the encoder and parameters of the transformer to minimize losses between (i) the first predicted frequency-domain representation and a first reference frequency-domain representation derived from the first modality and (ii) the second predicted frequency-domain representation and a second reference frequency-domain representation derived from the second modality.

In other features, the method includes fine tuning the encoder and transformer on labeled fine-tuning data and providing the fine tuned encoder and transformer for inference on input data. The input data corresponds to a modality different from a modality of the time-series input signal.

In other features, the method includes resampling the input data to match a sampling rate of the time-series input signal.

In other features, the method includes zero-padding resampled input data having a temporal length shorter than a temporal length of the time-series input signal.

In other features, the method includes dividing the resampled input data having a temporal length longer than a temporal length of the time-series input signal into a plurality of overlapping windows, processing each window using the encoder and the transformer to generate corresponding inference contextual features, and averaging the inference contextual features to generate an aggregated representation.

In other features, the encoded representation includes a subject-specific embedding, the method further includes adjusting the subject-specific embeddings to minimize the loss between the predicted frequency-domain representation and the reference frequency-domain representation derived from the time-series input signal.

In other features, the encoder includes a convolutional neural network configured to extract local temporal features from the time-series input signal to generate the encoded representation and the transformer includes a temporal self-attention model configured to extract global temporal features from the encoded representation to generate the contextual features.

Other examples provide a non-transitory computer-readable medium including executable instructions that, when executed by an electronic processor, causes the electronic processor to process a time-series input signal using an encoder to produce an encoded representation, segment the encoded representation into a plurality of patches, applying a masking operation to a subset of the patches to produce a masked encoded representation, process the masked encoded representation using a transformer to generate contextual features, process the contextual features using a decoder to generate a predicted frequency-domain representation of the time-series input signal, and adjust parameters of the encoder and parameters of the transformer to minimize a loss between the predicted frequency-domain representation and a reference frequency-domain representation derived from the time-series input signal.

Other examples provide a computer-implemented method that includes processing input data using an encoder to generate an encoded representation, processing the encoded representation using a transformer to generate contextual features, and processing the contextual features using an inference task head to generate inference results. The encoder and the transformer are pretrained using a time-series input signal by applying a masking operation to a subset of patches of a training encoded representation of the time-series input signal generated by the encoder, processing the masked encoded representation using the transformer to generate training contextual features, processing the training contextual features using a decoder to generate a predicted frequency-domain representation of the time-series input signal, and adjusting parameters of the encoder and parameters of the transformer to minimize a loss between the predicted frequency-domain representation and a reference frequency-domain representation derived from the time-series input signal.

In other features, the masking operation includes masking one or more consecutive sequences of fixed-sized patches from the training encoded representation.

In other features, the time-series input signal includes a first modality and a second modality, and the encoder and the transformer are pretrained by processing the training contextual features using a first decoder to generate a first predicted frequency-domain representation of the time-series input signal, processing the training contextual features using a second decoder to generate a second predicted frequency-domain representation of the time-series input signal, and adjusting parameters of the encoder and parameters of the transformer to minimize losses between (i) the first predicted frequency-domain representation and a first frequency-domain representation derived from the first modality and (ii) the second predicted frequency-domain representation and a second reference frequency-domain representation derived from the second modality.

In other features, the input data corresponds to a modality different from a modality of the time-series input signal.

In other features, the method includes resampling the input data to match a sampling rate of the time-series input data.

In other features, the method includes zero-padding resampled input data having a temporal length shorter than a temporal length of the time-series input signal.

In other features, the method includes dividing the resampled input data having a temporal length longer than a temporal length of the time-series input signal into a plurality of overlapping windows, processing each window using the encoder and the transformer to generate corresponding contextual representations, and averaging the contextual representations to generate the contextual features.

In other features, the encoded representation includes a subject-specific embedding, the subject-specific embedding learned during pretraining to minimize the loss between the predicted frequency-domain representation and the reference frequency-domain representation.

In other features, the encoder includes a convolutional neural network configured to extract local temporal features from the time-series input signal to generate the encoded representation and the transformer includes a temporal self-attention model configured to extract global temporal features from the encoded representation to generate the contextual features.

Other examples provide a system including non-transitory computer-readable storage media storing instructions and an electronic processor configured to execute the instructions. Executing the instructions causes the electronic processor to process input data using an encoder to generate an encoded representation, process the encoded representation using a transformer to generate contextual features, and process the contextual features using an inference task head to generate inference results. The encoder and the transformer are pretrained using a time-series input signal by applying a masking operation to a subset of patches of a training encoded representation of the time-series input signal generated by the encoder, processing the masked encoded representation using the transformer to generate training contextual features, processing the training contextual features using a decoder to generate a predicted frequency-domain representation of the time-series input signal, and adjusting parameters of the encoder and parameters of the transformer to minimize a loss between the predicted frequency-domain representation and a reference frequency-domain representation derived from the time-series input signal.

Other examples, embodiments, features, and aspects will become apparent by consideration of the detailed description and accompanying drawings.

### BRIEF DESCRIPTION OF THE DRAWINGS

FIG. 1 is a block diagram illustrating an example computing system that may be used to implement machine learning techniques for pretraining, deployment, and inference, according to some examples.

FIG. 2 is a block diagram schematically illustrating a model store, according to some examples.

FIG. 3 is a block diagram illustrating example data flow between an encoder and a transformer, according to some examples.

FIG. 4 is a block diagram schematically illustrating a model store, according to some examples.

FIG. 5 is a message sequence chart illustrating interactions between components of the system of FIG. 1, according to some examples.

FIG. 6 is a flowchart illustrating an example process for training a feature extractor, according to some examples.

FIG. 7 is a block diagram illustrating example data flow between an encoder, a transformer 206, and a decoder during a training process, according to some examples.

FIG. 8 is a block diagram illustrating example data flow between an encoder, a transformer, and pretraining heads during a training process, according to some examples.

FIG. 9 is a flowchart illustrating an example process for performing inference using a trained feature extractor, according to some examples.

FIG. 10 is a block diagram illustrating example data flow between an encoder, a transformer, and an inference head during an inference process, according to some examples.

In the drawings, reference numbers may be reused to identify similar and/or identical elements.

### DETAILED DESCRIPTION

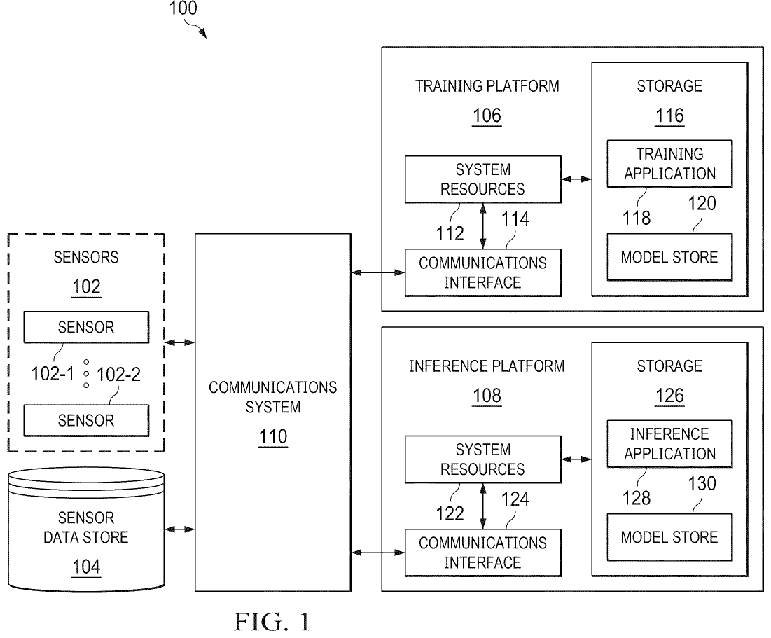

FIG. 1 is a block diagram illustrating an example computing system 100 that may be used to implement machine learning techniques for pretraining, deployment, and inference, according to some examples. The system 100 may include one or more sensors 102 (such as, for example, sensors 102-1 and 102-2), a sensor data store 104, a training platform 106, and an inference platform 108. The system 100 may also include a communications system 110 connecting the various sensors, data stores, and platforms of the system 100. For example, the sensors 102, sensor data store 104, training platform 106, and/or inference platform 108 may communicate with one another via the communications system 110. Although two sensors 102, a single sensor data store 104, a single training platform 106, and a single inference platform 108 are illustrated in the example of FIG. 1, other implementations of the system 100 may include any number of each sensor, data store, or platform.

The sensors 102 may include one or more sensors that generate sensor data from bio-signals. Examples of sensors 102 include any combination of EEG sensors, ECG sensors, EMG sensors, PPG sensors, EOG sensors, accelerometers, and/or any other suitable sensors. EEG sensors monitor the brain's electrical activity by capturing voltage fluctuations from the scalp using electrodes. These sensors generate multi-channel time-series data that reflect neural oscillations and frequency-domain characteristics such as alpha, beta, and theta rhythms. EEG data are widely used for applications such as cognitive state assessment, sleep stage classification, and seizure detection. ECG sensors measure the electrical activity of the heart, typically using electrodes placed on the chest or limbs. They produce time-series waveforms that include features such as P waves, QRS complexes, and T waves. These signals are used for arrhythmia detection, heart rate variability analysis, and biometric identification.

EMG sensors detect the electrical activity produced by skeletal muscles during contraction. They generate time-series data that contain muscle activation patterns, bursts, and resting phases. EMG data are commonly used in gesture recognition, prosthetic control, and neuromuscular disorder diagnostics. PPG sensors utilize optical methods—usually involving infrared or red LEDs and photodetectors—to measure blood volume changes in peripheral tissues. The resulting waveform reflects cardiovascular activity and can be used to derive heart rate, estimate blood oxygen saturation, and assess stress levels.

EOG sensors record eye movements by detecting the corneo-retinal potential between the front and back of the eye. These sensors produce signals indicative of eye blinks, saccades, and other ocular motion, making them useful in sleep studies, fatigue monitoring, and human-computer interaction applications. Accelerometers measure physical movement by detecting changes in velocity or orientation. These sensors typically produce tri-axial time-series data and are used for human activity recognition, posture classification, and motion analysis in wearable systems.

The sensors 102 may record sensor signals corresponding to physiological and/or movement-based activity. These signals may be acquired continuously or at defined intervals, and may represent raw or partially processed data from one or more channels. Depending on the sensor type, local operations such as amplification, filtering, or digitization may be applied before the signals are made available for further use. The recorded sensor signals may be transmitted from the sensors 102 to the sensor data store 104 (for example, via the communications system 110). Transmission may occur in real-time or in batches, depending on system configuration and application needs. In some implementations, signals are streamed as they are recorded; in others, data may be buffered and transmitted according to a schedule or triggered condition.

The sensor data store 104 may store the sensor signals in a variety of formats suited for time-series analysis. For example, sensor signals may be represented as multidimensional arrays (e.g., [channel×time]), tabular formats with timestamped rows of data, specialized time-series formats such as EDF, WFDB, HDF5, or XDF, and/or other suitable formats. These data formats may capture signal amplitude over time, along with structural attributes such as sampling rate, channel layout, and/or window boundaries.

In addition to the raw signal data, the sensor data store 104 may store metadata describing the conditions and context associated with each recording. This metadata may include subject-level information (e.g., identifier, demographic data, health status), acquisition parameters (e.g., sampling frequency, number of channels, sensor placement), and temporal indicators (e.g., timestamps, segment start times, event markers). Label information, where available, may include diagnostic annotations, physiological states, or behavioral conditions corresponding to the recorded data segments. Metadata may also include details about any preprocessing applied to the sensor signals, such as normalization, resampling, segmentation into patches, or augmentation.

The training platform 106 and the inference platform 108 may be implemented on various computing platforms. These platforms may include traditional computing systems such as desktop computers, laptops, workstations, and servers. In various implementations, the computing platforms may also include mobile computing devices, such as smartphones and tablets. The processing steps described herein may be performed on a single computing platform or distributed across multiple platforms, depending on the specific implementation needs.

The training platform 106 may include system resources 112, a communications interface 114, and non-transitory computer-readable storage media, such as storage 116. The non-transitory computer-readable storage media may contain instructions that, when executed, cause one or more electronic processors (for example, electronic processors of the system resources 112) to perform various functions described herein. The system resources 112 may include one or more electronic processors, graphics processing units, volatile and non-volatile computer memory, and system buses interconnecting various components of the training platform 106. The communications interface 114 may include hardware and/or software components that facilitate communication with other devices, platforms, and systems over the communications system 110. The communications interface 114 may include one or more transceivers for sending and receiving data over the communications system 110.

The storage 116 may include a training application 118 and a model store 120. The training application 118 may train machine learning models stored in the model store 120 according to techniques described herein and/or deploy the trained models to the inference platform 108, for example, via the communications system 110.

The inference platform 108 may include system resources 122, a communications interface 124, and non-transitory computer-readable storage media, such as storage 126. The non-transitory computer-readable storage media may contain instructions that, when executed, cause one or more electronic processors (for example, electronic processors of the system resources 122) to perform various functions described herein. The system resources 122 may include one or more electronic processors, graphics processing units, volatile and non-volatile computer memory, and system buses interconnecting various components of the inference platform 108. The communications interface 124 may include hardware and/or software components that facilitate communication with other devices, platforms, and systems over the communications system 110. The communications interface 124 may include one or more transceivers for sending and receiving data over the communications system 110.

The storage 126 may include an inference application 128 and a model store 130. The inference application 128 may receive trained machine learning models from the training platform 106, store received machine learning models at the model store 130, and/or perform inference using machine learning models stored at the model store 130, for example, according to techniques described herein.

In various implementations, the communications system 110 includes one or more types of networks to facilitate connectivity and data transmission. These may include mobile networks such as General Packet Radio Service (GPRS), Time-Division Multiple Access (TDMA), Code-Division Multiple Access (CDMA), Global System of Mobile Communications (GSM), Enhanced Data Rates for GSM Evolution (EDGE), High-Speed Packet Access (HSPA), Evolved High-Speed Packet Access (HSPA+), Long Term Evolution (LTE), Worldwide Interoperability for Microwave Access (WiMAX), and 5th-generation mobile networks (5G). Additionally, the communications system 110 may incorporate an Internet Protocol (IP) network, a Wireless Application Protocol (WAP) network, or an IEEE 802.11 standards network, as well as any suitable combination of these networks.

The communications system 110 may also include other network types, such as optical networks, local area networks (LANs), and global communication networks like the Internet. In some implementations, the communications system 110 may be implemented according to one or more serial communication standards, including RS-232, RS-485, Universal Asynchronous Receiver/Transmitter (UART), Inter-Integrated Circuit (I2C), Serial Peripheral Interface (SPI), and Universal Serial Bus (USB). Furthermore, the communications system 110 may include a Controller Area Network (CAN). In various implementations, the communications system 110 includes personal area networks (PANs) such as Bluetooth and Zigbee, allowing for short-range, wireless communication.

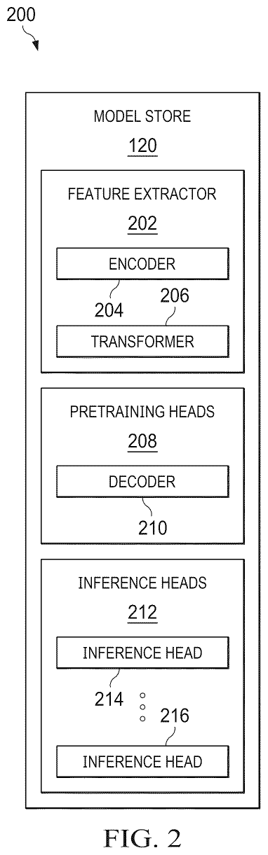

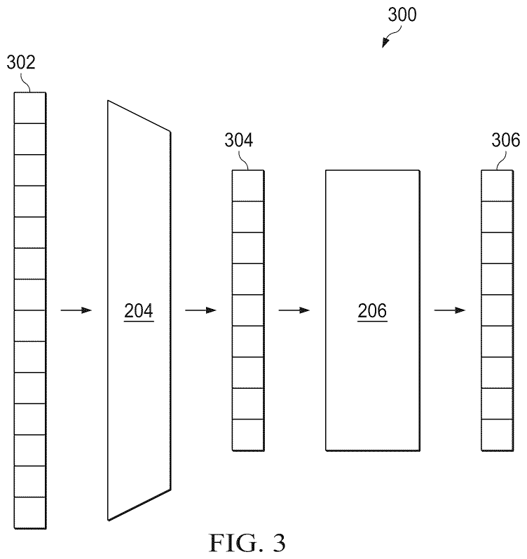

FIG. 2 is a block diagram 200 schematically illustrating the model store 120, according to some examples. In the example of FIG. 2, the model store 120 includes a machine learning model for extracting features from one or more bio-signal modalities, such as, for example, a feature extractor 202. The feature extractor 202 may include an encoder 204 and a transformer 206. FIG. 3 is a block diagram 300 illustrating example data flow between the encoder 204 and the transformer 206, according to some examples.

The feature extractor 202 may receive an input signal 302. In various implementations, the input signal 302 may be a time-series signal representing a bio-signal acquired from any of the sensors 102 and/or retrieved from the sensor data store 104 (e.g., via the communications system 110). For example, the input signal 302 may correspond to sensor signals generated by EEG sensors, ECG sensors, EMG sensors, PPG sensors, EOG sensors, accelerometers, or other suitable bio-signal acquisition devices. Different bio-signal modalities may capture different physiological or behavioral phenomena, such as brain activity (e.g., EEG), cardiac rhythms (e.g., ECG), muscle activation (e.g., EMG), blood volume changes (e.g., PPG), eye movements (e.g., EOG), or physical motion (e.g., accelerometer-based signals).

To prepare sensor signals for feature extraction, the training application 118 (during pretraining) and/or the inference application 128 (during inference) may perform one or more preprocessing operations. Preprocessing may include amplification, analog-to-digital conversion, filtering (for example, bandpass filtering to remove noise and artifacts), normalization (e.g., z-scoring or min-max scaling), resampling to a target sampling rate, segmentation into fixed-length time windows, and/or formatting into standardized array or tensor structures. In various implementations, preprocessing includes additionally restructuring multi-channel signals into a channel-independent format by concatenating individual sensor channels along the batch dimension. This approach allows the encoder 204 to process one channel at a time, facilitating channel-independent feature extraction.

Following preprocessing, the preprocessed sensor signals may be represented as the input signal 302. Structurally, the input signal 302 may take the form of a one-dimensional array for single-channel data, a two-dimensional array for multi-channel data (e.g., [channels×time]), or a higher-dimensional tensor when additional contextual information (e.g., metadata or auxiliary features) is included. The input signal 302 may vary in sampling rate, temporal length, number of channels, and amplitude range, depending on the originating sensor modality and application context. For example, EEG data may be sampled in a range of between about 100-1000 Hz, PPG data at about 64 Hz, and accelerometer data at about 50 Hz. In some examples, the input signal 302 may thus be raw or minimally processed aside from the preprocessing steps to standardize the format for input into the feature extractor 202.

In various implementations, the encoder 204 receives the input signal 302 and processes the input signal 302 to generate an encoded representation 304. The encoded representation 304 may represent relevant features of the input signal 302 transformed into a lower-dimensional latent space. In various implementations, “lower-dimensional latent space” may refer to a feature space where the temporal resolution is reduced relative to the original signal, and each element (e.g., patch) captures enriched representations of local temporal patterns such as transient bursts, oscillatory waveforms, or morphological characteristics of physiological activity. This compact encoding may facilitate efficient modeling and downstream analysis while preserving physiologically relevant information.

Architecturally, the encoder 204 may be implemented as a multi-layer CNN configured to extract hierarchical representations of temporal structures. At a high level, each successive layer of the CNN progressively transforms the input signal by (i) increasing the feature dimensionality (i.e., the number of output channels), (ii) reducing the temporal resolution by downsampling, and (iii) learning increasingly abstract and temporally extended features. Early layers of the CNN may capture simple localized patterns, while deeper layers may capture complex temporal interactions. Residual connections within each convolutional block facilitate information flow, enabling stable training dynamics and improved feature learning.

In some examples, the encoder 204 may include a three-layer residual convolutional network comprising a series of residual blocks. Formally, the encoder 204 may transform an input signal according to Equation (1):

$$C N N ( x ) ∈ {ℝ}^{D \times P} \qquad ( 1 )$$

In Equation (1), x∈ [symbol] ^{1×T} represents the input signal 302, T denotes the temporal length (e.g., the number of time steps or samples) of the input signal, D denotes the number of output feature channels (e.g., the feature dimensionality extracted by the CNN), and P denotes the number of temporal patches (e.g., the number of reduced-length segments output by the encoder 204).

Equation (1) may thus represent how the encoder 204 processes a one-dimensional input time-series into a two-dimensional latent representation, where each row corresponds to a learned feature channel and each column corresponds to a temporal patch or receptive field over the input. As the encoder 204 processes the input signal, the feature dimensionality D generally increases across layers (e.g., capturing richer information), while the temporal resolution is reduced to P (e.g., through downsampling operations), summarizing local temporal patterns into compressed patches.

The stride of the CNN layers may determine the spacing between adjacent patches, meaning how much the window moves across the input at each step. The receptive field of the CNN may determine the effective temporal duration covered by each patch, corresponding to how many consecutive input samples influence each output feature. Each patch thus encodes localized temporal features—for example, a burst of neural oscillations, a heartbeat segment, or a muscle contraction phase—into a compact, learned representation within the encoded feature space.

Structurally, the encoded representation 304 output by the encoder 204 may be represented as a matrix with shape D×P. In this matrix, each of the P columns correspond to a localized temporal segment (patch) of the original input signal 302, and each of the D rows corresponds to a different learned feature extracted by the CNN. Each element of the matrix thus encodes a specific feature response for a given temporal region. This patch-based, feature-rich representation may facilitate flexible manipulation for downstream operations, such as masking, transformer-based modeling, and reconstruction in the frequency domain.

Each patch in the encoded representation 304 may correspond to a specific receptive field over the original input signal 302, representing localized temporal information. Because each receptive field may capture a different segment of the input signal, each patch effectively encodes a filtered frequency spectrum corresponding to a specific temporal region. As a result, meaningful temporal relationships exist between patches for example, sequential patches may capture oscillatory patterns or transitions between physiological states.

To model these temporal dependencies between patches, the system 100 may the encoded representation 304 using a transformer 206, which applies self-attention mechanisms to learn relationships across patches based on their contextual similarity and temporal structure.

Each residual block in the encoder 204 may include two parallel computational paths. One path may comprise a single convolutional layer with a kernel size of 3, a stride of 2, and padding of 1, configured to increase the number of channels from C to 2C. The other path may comprise two sequential convolutional layers: a first convolutional layer with a kernel size of 3, a stride of 1, and padding of 1, configured to increase the number of channels from C to 2C, followed by a second convolutional layer with a kernel size of 3, a stride of 2, and padding of 1, configured to maintain the number of output channels. In various examples, the convolutional layers may omit bias parameters to promote parameter regularization.

After the convolutional operations, the output of each convolutional layer may be followed by a batch normalization (BatchNorm) operation and a GELU activation function. Between the two convolutional layers in the two-layer path, a Dropout layer may also be applied to promote regularization. After both paths are processed, their outputs may be summed to implement the residual connection, and the combined output may be passed through an additional GELU activation and Dropout layer. This residual structure helps maintain information flow through the network while enabling the modeling of non-linear and temporally complex features. Throughout training and fine-tuning, a Dropout probability of 0.1 may be applied within the encoder 204 to reduce the risk of overfitting.

In various implementations, the transformer 206 may receive the encoded representation 304 as input and process the encoded representation 304 to generate contextual features 306 as output. Broadly, a transformer network may be a neural network architecture designed to model complex relationships between inputs using self-attention mechanisms. Transformers can capture both local and global dependencies in sequential data, making them highly effective for tasks such as language modeling, image processing, and time-series analysis. In the context of bio-signal modeling, transformers may be particularly effective for learning long-range temporal relationships, physiological rhythms, and dynamic state transitions that may not be captured effectively using local convolutional operations alone.

The transformer 206 may be implemented according to a Patch Time Series Transformer (PatchTST) architecture. In PatchTST, rather than processing individual time steps independently, the input sequence may be divided into patches, where each patch may be a contiguous segment of the original input signal. These patches may be treated as discrete tokens, and self-attention mechanisms are applied across the patches to model their interrelationships. By operating on patches rather than individual samples, PatchTST improves computational efficiency, reduces sequence length, and enhances the model's ability to simultaneously capture short-term dynamics within patches and long-term dependencies across patches. This patch-based strategy enables the model to flexibly integrate localized and global temporal information, which may be particularly beneficial for modeling the complex, multiscale nature of bio-signals.

In some examples, the transformer 206 may include several sequential processing stages. First, the encoded patches from the encoded representation 304 may be passed through a patch embedding layer, which may apply a learnable linear projection to map each patch into a fixed-size embedding space. This transforms the sequence of patches into a sequence of dense feature vectors. Positional encodings may then be added to the patch embeddings to incorporate information about the temporal order of patches, allowing the transformer to maintain awareness of sequence structure. The positional encodings may be learned during training or predefined (for example, using sinusoidal functions). The embedded patches with positional information may be processed through one or more transformer encoder layers, where each encoder layer may include a multi-head self-attention mechanism to learn relationships between patches, a feedforward neural network (FFN) to refine and transform features at each position, normalization layers (such as layer normalization) to stabilize learning, and residual connections to preserve feature information and promote efficient training. Dropout operations may also be applied after attention and feedforward operations to enhance regularization and reduce overfitting.

Through these operations, the transformer 206 may process the input sequence of patch embeddings to generate a contextually enriched output, where each patch representation incorporates both local patch-level features and global context aggregated from the entire input sequence. The self-attention mechanism enables flexible, data-driven modeling of temporal dependencies across patches, allowing the model to capture complex interactions between localized events and broader temporal trends that unfold across extended time windows.

The transformer 206 may output the contextual features 306. The contextual features 306 may represent a temporally-aware, globally-informed encoding of the input signal 302, capturing both fine-grained temporal structures (such as transient oscillations or event onsets) and broader physiological patterns (such as sustained changes in state or rhythm). Structurally, the contextual features 306 may be represented as a matrix with shape D′×P, where D′ denotes the dimensionality of the transformed feature space produced by the transformer 206, and P denotes the number of patches corresponding to temporal segments of the original input signal 302. In this representation, each column of the contextual features 306 corresponds to a temporally localized region of the input, enriched with information from the full temporal context. The contextual features 306 may be used for a variety of downstream operations, such as reconstructing masked portions of the input signal, predicting frequency-domain representations, or performing supervised tasks such as classification or anomaly detection.

The model store 120 may include one or more pretraining heads, such as pretraining heads 208. The pretraining heads 208 may include a decoder 210 configured to decode the contextual features 306 output by the transformer 206 and generate a predicted output corresponding to the input signal 302. Broadly, a decoder network in this context refers to a neural network component designed to invert or reconstruct representations learned by the encoder and transformer. The decoder 210 may be trained to reconstruct certain target features from masked or partially observed inputs, thereby encouraging the model to learn structured, generalizable representations of the underlying physiological signals.

In some examples, the decoder 210 may be configured to output a predicted frequency-domain representation of the input signal 302. For instance, the decoder 210 may reconstruct a time-frequency representation such as a spectrogram, a Mel spectrogram, a power spectral density (PSD), a short-time Fourier transform (STFT), or another suitable time-frequency decomposition corresponding to masked portions of the input. In various implementations, the spectrogram may be further processed by z-scoring along the time axis, normalizing each spectral bin to have zero mean and unit variance over time. This z-scored spectrogram emphasizes learning non-trivial spectral patterns that persist across patches, rather than trivial absolute amplitude variations. Reconstructing frequency-domain representations during pretraining offers several technical advantages: frequency-domain structures in physiological signals—such as rhythmic oscillations, burst activity, and spectral peaks—tend to be more consistent across different subjects, sessions, and devices than time-series waveforms. As a result, training the model to predict frequency-domain outputs may improve generalization across recording conditions, modalities, and individuals, enhancing cross-subject and cross-modal transferability.

The decoder 210 may be implemented using one or more neural network layers, such as fully connected (dense) layers, convolutional layers, transposed convolution (deconvolution) layers, or other suitable structures. In one example, the decoder 210 may apply a sequence of linear transformations and non-linear activations to progressively transform the contextual features 306 into the desired output format. The decoder 210 may upsample or interpolate the contextual features as needed to match the resolution of the target frequency-domain output. In some implementations, the decoder 210 may mirror the structure of the encoder—for example, by using a sequence of transposed convolutional layers arranged in a residual or “flipped” architecture—to reconstruct higher-resolution outputs from the lower-resolution contextual features. Additionally, normalization layers, dropout layers, and residual connections may be incorporated into the decoder architecture to promote stable training and enhance generalization performance.

Although the examples described herein primarily illustrate the decoder 210 generating frequency-domain outputs, other implementations are possible. For example, in some cases, the decoder 210 may be configured to reconstruct the original time-series waveform of the input signal 302 instead of, or in addition to, a frequency-domain representation. In these examples, the decoder 210 may directly predict masked or corrupted segments of the raw time-series signal. Time-domain reconstruction may be particularly beneficial for tasks requiring precise temporal fidelity, such as denoising, interpolation, signal completion, or artifact removal. In some implementations, frequency-domain and time-domain reconstruction objectives may be combined during training to encourage the model to learn complementary information across both representations.

Furthermore, although a single decoder 210 is illustrated in the example of FIG. 2, other implementations may include any number of decoders. For example, different decoders may be provided for different modalities (e.g., EEG, ECG, EMG, PPG) or for different output types (e.g., time-domain waveform, frequency-domain spectrogram, or other task-specific targets). In multimodal settings, contextual features corresponding to each modality may be processed separately by dedicated decoders specialized for reconstructing the appropriate output type. Each decoder may thus be tailored for the specific characteristics and pretraining objectives associated with its corresponding input domain or signal modality.

In various implementations, the model store 120 may also include one or more inference heads, such as inference heads 212. Inference heads 212 may be configured to receive the contextual features 306 output by the feature extractor 202 and generate task-specific outputs suitable for downstream inference tasks. Each inference head may map the contextual features 306 to outputs appropriate for a given application, such as classification, regression, segmentation, or anomaly detection.

For example, the inference heads 212 may include a first inference head 214 and a second inference head 216. Although two inference heads are illustrated in the example of FIG. 2, other implementations may include any number of inference heads, as may be suitable for the particular application needs. Different inference heads may be specialized for different target modalities, output formats, or types of tasks. In some examples, an inference head may be designed to classify physiological states (e.g., sleep stages, cognitive workload levels, arrhythmia classes) based on bio-signal data. In other examples, an inference head may perform regression tasks, such as predicting continuous physiological variables (e.g., heart rate, respiratory rate, blood oxygen saturation) from the input signals. Still other examples may involve multi-label classification, temporal segmentation of signals, or detection of abnormal or anomalous patterns.

Structurally, an inference head may include one or more neural network layers suitable for transforming the contextual features 306 into task-specific outputs. Suitable inference head architectures may include, for example, one or more fully connected (dense) layers followed by an output layer, such as a softmax layer for multi-class classification, a sigmoid layer for binary or multi-label classification, or a linear output layer for regression tasks. In some examples, additional operations such as batch normalization, dropout regularization, or residual connections may be incorporated into the inference head to improve stability and performance. In other examples, more complex inference heads may include attention mechanisms, recurrent layers (e.g., LSTMs, GRUs), or temporal convolutional layers to further refine temporal dependencies in the contextual features before output generation.

During deployment, the inference application 128 may use one or more inference heads to perform task-specific inference based on the contextual features 306 generated by the feature extractor 202. In some examples, different inference heads may be selected or switched dynamically based on the application context, the type of input modality, or the specific inference task to be performed. By modularly combining a shared feature extractor 202 with multiple specialized inference heads 212, the system 100 may flexibly adapt to a wide range of use cases, modalities, and signal types while leveraging a common pretrained feature space.

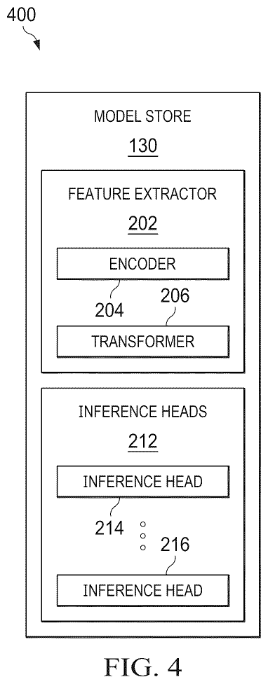

FIG. 4 is a block diagram 400 schematically illustrating the model store 130, according to some examples. In various implementations, the training application 118 may train the feature extractor 202 and/or one or more inference heads 212. After training is completed, the training application 118 may deploy the trained feature extractor 202 and/or inference heads 212 to the inference platform 108. For example, the training application 118 may transmit the trained feature extractor 202 and/or inference heads 212 to the inference platform 108, where the inference application 128 stores the received feature extractor 202 and/or inference heads 212 in the model store 130. In some implementations, the training application 118 may transmit model parameters—such as learned weights, biases, and configuration metadata—corresponding to the feature extractor 202 and/or the inference heads 212, and the inference application 128 may reconstruct the feature extractor 202 and/or the inference heads 212 on the inference platform 108 based on the received parameters.

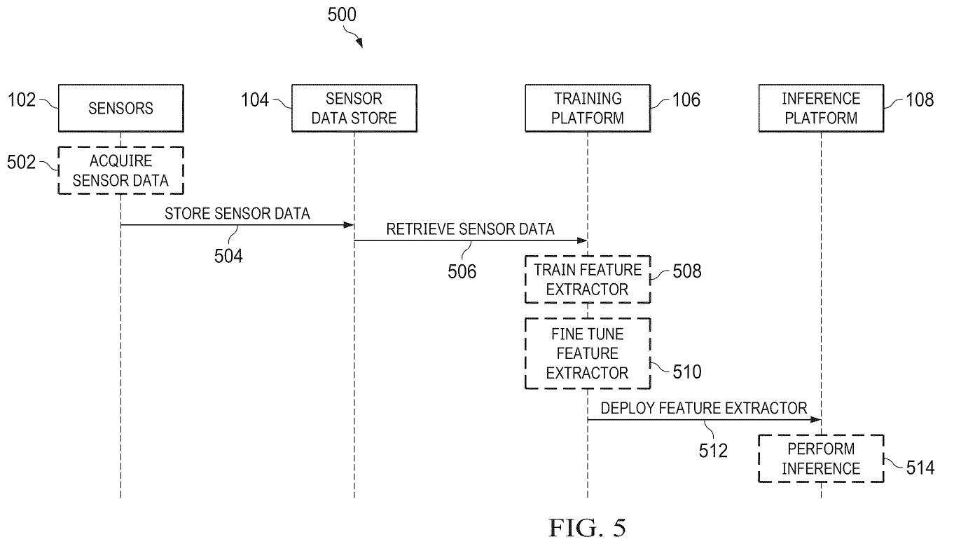

FIG. 5 is a message sequence chart 500 illustrating interactions between components of the system 100, according to some examples. The example message chart 500 illustrates how the system 100 acquires sensor data of a first modality (such as EEG data), pretrains a machine learning model using this available sensor data, and subsequently deploys the pretrained model to an inference platform 108. This pretraining may facilitate inference applications that can process either the original first modality or, importantly, sensor data from different modalities (such as EMG, ECG, or PPG), leveraging the cross-modal generalization capabilities described earlier. The sequence illustrates how the system 100 may address the technical challenge of data scarcity in certain bio-signal domains by transferring knowledge from data-rich modalities to those with limited available training data.

In the example message sequence chart 500, the sensors 102 acquire sensor data (at operation 502). For example, the sensors 102 may be any of the previously described sensors and acquire sensor data according to any of the previously described techniques. In the example message sequence chart 500, the sensors 102 store the acquired sensor data at the sensor data store 104 (at operation 504). In various implementations, the sensor data is processed according to any of the previously described techniques. In the example message sequence chart 500, the training platform 106 retrieves the sensor data from the sensor data store 104 (at operation 506). For example, the training application 118 may retrieve available sensor data corresponding to one or more bio-signal modalities from the sensor data store 104. Examples of retrieved sensor data may include EEG data, EMG data, motion sensor data, epilepsy-related EEG data, machine condition monitoring data, PPG data, human activity recognition (HAR) data, and ECG data.

EEG data may include time-series recordings sampled at approximately 100 Hz, segmented into 30-second windows. In some cases, the EEG data may include signals from two channels placed on the scalp, capturing brain electrical activity. Single-channel electrooculography (EOG) recordings sampled at a similar rate may also be retrieved to supplement the EEG signals, providing additional information about eye movements. The training application 118 may process the EEG and/or EOG signals either independently or jointly, depending on the pretraining or downstream task objectives.

EMG data may include single-channel recordings of skeletal muscle electrical activity sampled at approximately 4000 Hz, segmented into windows of approximately 375 milliseconds. These recordings may capture transient bursts associated with muscle contractions. The training application 118 may process the EMG signals to extract localized motor patterns, spectral signatures, or activation dynamics relevant to gesture recognition or neuromuscular disorder detection.

Motion sensor data may include tri-axial accelerometer recordings sampled at approximately 100 Hz, segmented into windows of approximately 3.15 seconds. The data may include three channels corresponding to acceleration measurements along orthogonal axes. The training application 118 may process the motion signals to identify patterns of movement, gestures, or postural transitions suitable for applications such as gesture recognition or physical activity classification.

Epilepsy-related EEG data may include single-channel brain activity recordings sampled at approximately 174 Hz, segmented into windows of approximately 1.02 seconds. These signals may capture both normal brain rhythms and pathological events such as epileptiform discharges. The training application 118 may process the epilepsy-related EEG data to extract temporal patterns indicative of seizure activity or other neurological conditions.

Machine condition monitoring data may include high-frequency recordings sampled at approximately 64,000 Hz, segmented into windows of approximately 80 milliseconds. These signals may capture vibrational or acoustic signatures from mechanical systems, such as electric motors or industrial machinery. The training application 118 may process these recordings to detect patterns associated with normal operation or early-stage mechanical faults.

PPG data may include single-channel optical pulse waveform recordings sampled at approximately 64 Hz, segmented into windows of approximately 60 seconds. The PPG signals may reflect blood volume changes in peripheral tissues. The training application 118 may process the PPG signals to extract features such as heart rate, pulse morphology, and heart rate variability, supporting applications in cardiovascular monitoring and affective state detection.

HAR data may include multi-channel accelerometer recordings sampled at approximately 50 Hz, segmented into windows of approximately 2.56 seconds. In some examples, the HAR data may include six channels corresponding to tri-axial accelerometer measurements from multiple sensor locations on the body. The training application 118 may process the HAR signals to classify physical activities, detect locomotion patterns, or infer postural transitions.

ECG data may include two-channel electrocardiographic recordings sampled at approximately 250 Hz, segmented into windows of approximately 10 seconds. These signals may capture cardiac electrical activity along different lead axes, including characteristic features such as P waves, QRS complexes, and T waves. The training application 118 may process the ECG signals to extract temporal intervals and morphological patterns relevant for arrhythmia detection, biometric authentication, or heart rate variability analysis.

In various implementations, the training application 118 may process the retrieved sensor data using the original window durations as described above. However, for certain downstream tasks where physiological events unfold over shorter timescales, the training application 118 may re-segment the data into shorter, standardized windows for example, into 2-second segments. This re-segmentation may enhance model performance for tasks requiring finer temporal resolution while maintaining consistency across different bio-signal types during pretraining and fine-tuning.

To facilitate robust model evaluation and minimize performance variance, the training platform 106 may implement a cross-validation procedure. In some examples, the available dataset may be partitioned into ten folds, with each fold serving as a test set once while the remaining folds are used for training and validation. In implementations simulating limited-data scenarios, the training and validation folds may be subsampled to a specified data regime (e.g., about 5%, about 10%, or about 25% of the available data). Within the subsampled data, about 75% may be allocated for training and about 25% for validation. Model performance may then be averaged across multiple random seeds and cross-validation splits to provide a comprehensive and reliable assessment of generalization across both high- and low-data regimes.

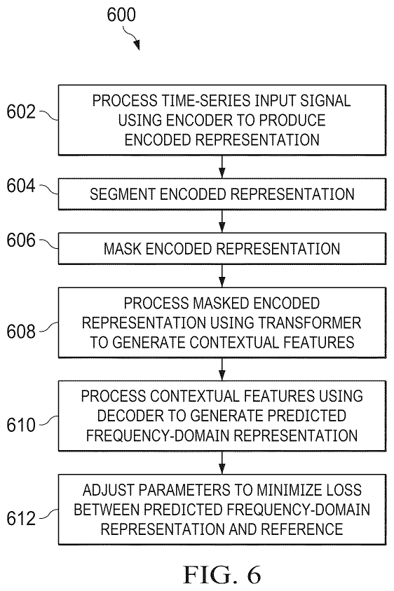

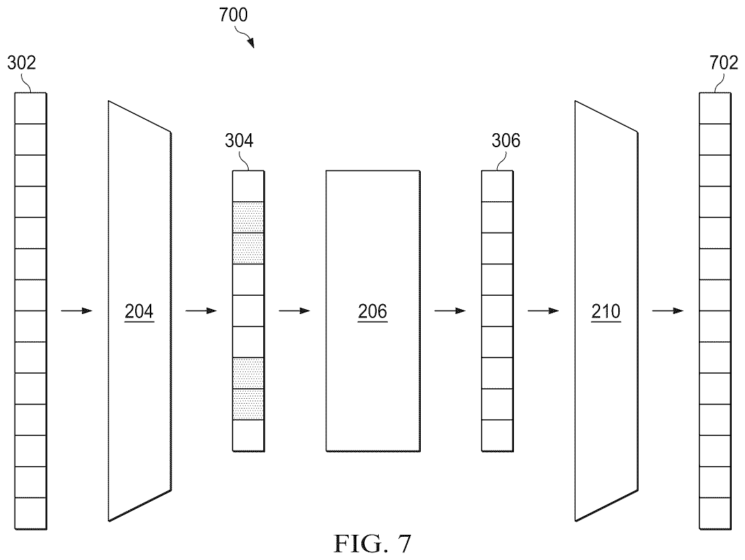

In the example message sequence chart 500, the training application 118 trains the feature extractor 202 using the retrieved and/or processed sensor data (at operation 508). FIG. 6 is a flowchart illustrating an example process 600 for training the feature extractor 202, according to some examples. FIG. 7 is a block diagram 700 illustrating example data flow between the encoder 204, the transformer 206, and the decoder 210 during the training process, according to some examples. Referring collectively to FIGS. 6 and 7, in the example process 600, the training application 118 processes the time series input signal 302 using the encoder 204 to produce the encoded representation 304 (at block 602), for example, according to any of the previously described techniques.

In the example process 600, the training application 118 segments the encoded representation 304 into a plurality of fixed-size, non-overlapping patches of equal length (at block 604). Following segmentation, the training application 118 applies a masking operation to a subset of the patches (at block 606). In various implementations, the training application 118 randomly selects one or more starting positions within the sequence of patches and masks contiguous sequences of patches beginning at the selected positions. The length of each masked sequence may be fixed (for example, masking eight consecutive patches per sequence), but the starting positions may be selected randomly across the encoded representation 304.

This block-wise random masking strategy addresses the redundancy that arises from overlapping receptive fields in the input signal 302. When the encoder 204 is implemented as a CNN, each patch in the encoded representation 304 corresponds to a receptive field over the original time-series input signal 302 that significantly overlaps with the receptive fields of neighboring patches. As a result, adjacent patches may encode highly correlated or redundant information.

Masking only isolated patches may allow unmasked neighboring patches to reveal much of the masked content, limiting the effectiveness of the masking objective. By instead masking longer contiguous sequences of patches, the model is forced to reason over larger temporal spans and infer missing content from more distant context. This encourages the model to learn broader temporal and frequency-domain structures rather than relying on short-range redundancy, thereby improving the robustness and generalizability of the learned features across different bio-signal modalities.

In the example process 600, the training application 118 provides the masked encoded representation 304 to the transformer 206 (at block 608). The transformer 206 processes the masked encoded representation 304 to generate the contextual features 306, for example, according to any of the techniques previously described. The masked encoded representation 304 includes one or more sequences of patches, where each patch represents a receptive field segment over the original input signal 302 generated by the encoder 204. Masked patches are replaced with a learnable mask token. Because contiguous sequences of patches are masked at random positions, the transformer 206 cannot rely solely on local neighborhood information. Instead, the transformer 206 must use self-attention mechanisms to integrate information across non-masked patches over extended temporal spans, capturing long-range dependencies and global frequency-domain relationships.

This masking forces the transformer 206 to infer missing content by reasoning about the underlying frequency structure of the signal—such as periodic rhythms, harmonics, and spectral continuity—rather than simply interpolating missing segments based on short-range similarity. As a result, the contextual features 306 produced by the transformer 206 encode not only local temporal features but also global, modality-agnostic frequency-domain structures. These representations are more robust to variations across subjects, sessions, and modalities and support generalization to new bio-signal domains where labeled data is scarce. By encouraging frequency-domain reconstruction during pretraining, the system improves cross-modal transferability and downstream performance.

In the example process 600, the training application 118 provides the contextual features 306 to the decoder 210. The decoder 210 processes the contextual features 306 to generate a predicted frequency-domain representation 702, for example, according to any of the techniques previously described (at block 610).

In various implementations, the decoder 210 reconstructs a predicted time-frequency representation of the input signal 302, such as a spectrogram, a Mel spectrogram, a power spectral density (PSD) map, or a short-time Fourier transform (STFT). The generated frequency-domain representation 702 may represent the distribution of spectral energy over time, capturing key physiological rhythms, oscillations, and transient spectral bursts that characterize bio-signals such as EEG, ECG, EMG, and PPG.

In some examples, the predicted frequency-domain representation 702 is further processed by z-scoring along the time axis. Z-scoring normalizes each frequency bin to have zero mean and unit variance across time, emphasizing relative spectral fluctuations while suppressing absolute amplitude biases that may vary across recording sessions, subjects, or devices. This normalization encourages the model to focus on learning intrinsic spectral structures—such as relative power distributions, frequency band activations, and spectral continuity patterns—rather than memorizing trivial amplitude information.

Generating and reconstructing a frequency-domain representation, rather than a raw temporal waveform, provides several technical advantages. Frequency-domain structures in bio-signals tend to be more stable, interpretable, and modality-invariant compared to time-domain waveform shapes, which can be highly variable across individuals, sessions, and recording conditions. Physiological processes such as neural oscillations, cardiac cycles, muscle activations, and hemodynamic rhythms manifest consistently in the frequency domain, often with characteristic spectral signatures that persist across subjects and devices.

By training the model to predict masked portions of the frequency-domain representation, the system forces the model to infer missing spectral information from available contextual cues. This pushes the model to reason about global frequency relationships, such as spectral peaks, harmonic structures, inter-band correlations, and continuity across frequency bands, rather than relying on localized temporal patterns. The masking of contiguous patch sequences at random positions further prevents the model from trivially reconstructing missing regions based on short-range temporal redundancy, thereby encouraging the development of deeper, modality-agnostic representations.

As a result, pretraining the machine learning model with a masked frequency-domain reconstruction objective improves its ability to capture long-range temporal and spectral dependencies, enhances robustness to variations in signal acquisition conditions (such as sensor type, noise levels, or subject-specific morphology), and enables effective cross-modal transfer across different bio-signal types. The pretrained model may generalize to new domains with minimal fine-tuning, providing significant advantages for downstream tasks where high-quality labeled training data is limited or unavailable. Furthermore, frequency-domain pretraining improves cross-subject robustness, helping the system maintain performance when deployed across diverse populations without requiring extensive retraining.

In the example process 600, the training application 118 adjusts parameters of the encoder 204 and/or transformer 206 to minimize the reconstruction loss between the predicted frequency-domain representation 702 and a corresponding reference frequency-domain representation of the input signal (at block 612). The reference frequency-domain representation may be generated by applying a time-frequency decomposition—such as a short-time Fourier transform (STFT)—to the input signal 302 to produce a spectrogram, spectrograph, or other time-frequency representation structurally matched to the predicted output (e.g., the predicted frequency-domain representation 702). In some implementations, both the predicted and reference frequency-domain representations are further processed by z-scoring along the time axis, normalizing each frequency bin to have zero mean and unit variance over time. This normalization emphasizes relative spectral variations while minimizing the impact of absolute amplitude differences, promoting more robust learning of intrinsic frequency structures.

The reconstruction loss may be computed using one or more suitable loss functions, including mean squared error (MSE), mean absolute error (MAE), smooth L1 loss (Huber loss), Kullback-Leibler (KL) divergence, or cosine similarity loss. In certain implementations, the total loss may be formulated as a weighted combination of multiple individual loss terms, with adjustable weights (e.g., λ₁ for MSE, λ₂ for KL divergence) tuned according to training objectives. The training application 118 may dynamically select and adjust these loss hyperparameters based on performance metrics such as validation loss stability, frequency reconstruction fidelity, or cross-modal generalization performance.

After computing the total reconstruction loss, the training application 118 adjusts parameters of the encoder 204 and/or transformer 206 based on the computed gradients. For example, the training application 118 may compute gradients of the reconstruction loss with respect to learnable parameters and apply an optimization algorithm to update the parameters to minimize the loss.

Suitable optimizers may include adaptive gradient-based methods such as Adam, AdamW, RMSProp, or Lookahead, with configurable hyperparameters such as learning rate, beta values for momentum estimation, weight decay coefficients, and epsilon values for numerical stabilization. In various implementations, dynamic learning rate scheduling—such as cosine annealing, one-cycle policies, or stepwise decay—may be employed, with tunable parameters controlling warm-up steps, minimum learning rates, and annealing cycles.

Additionally, training hyperparameters related to the masking strategy—such as the proportion of patches masked, the length of masked contiguous sequences, and random seeds controlling masking variability—may be tuned to balance task difficulty and training efficiency. Through iterative updates of model and optimizer parameters based on the masked reconstruction loss, the encoder 204 and transformer 206 progressively learn latent feature representations that encode temporally extended and spectrally coherent structures, improving generalization across different physiological modalities.

In various implementations, the feature extractor 202 may incorporate a learnable subject-specific embedding to encode individual-specific characteristics and allow the feature extractor 202 to adapt across different subjects or sensor configurations. Physiological signals, such as electroencephalography (EEG) recordings, can be influenced by subject-dependent factors including anatomical variations (e.g., head size), electrode placement, or skin conductivity. Rather than attempting to eliminate these effects during preprocessing, the system accounts for such variability during model training by introducing subject-specific embeddings at the feature level.

In transformer-based architectures, such as the transformer 206, positional embeddings are typically added to input patches to encode temporal ordering and patch relationships. These positional embeddings may be pre-defined or learned during training. Similarly, in the techniques described herein, a subject-specific embedding is added to each patch to encode subject identity. However, instead of being unique for each patch (as with positional embeddings), the subject-specific embedding is unique for each subject represented in the training batch.

For example, the patches P of the segmented encoded representation 304 may be represented as Equation (2) below:

$$P ∈ {R}^{S \times N \times D} \qquad ( 2 )$$

In Equation (2), S denotes the number of subjects in the batch, N denotes the number of patches, and D denotes the feature dimensionality of each patch. For each subject s∈{1, . . . , S}, a subject-specific embedding vector e_{subject,s}∈R^{S×D} may be associated with that subject. The subject-specific embedding may be broadcast across all patches corresponding to the subject and added to the encoded patches as represented by Equation (3) below:

$${P}_{s} = {P}_{s} + {e}_{subject , s} \qquad ( 3 )$$

The subject-specific embeddings may be initialized randomly and trained jointly with the model parameters using backpropagation. During pretraining or fine-tuning, the embeddings are updated to minimize the overall loss function, allowing the model to learn subject-specific offsets that improve reconstruction fidelity or downstream task performance. In some implementations, the embeddings may be optionally fixed or pre-computed based on known subject metadata (e.g., demographic information or device calibration parameters), although in many cases the embeddings are learned purely from the data without external supervision.

In scenarios where all training data corresponds to a single subject or a single consistent sensor configuration, the subject-specific embedding reduces to a single vector shared across all patches. In such cases, the embedding acts as a constant offset and may either be retained as a learnable parameter or omitted entirely without substantial impact on model performance. Thus, the subject-specific embedding mechanism introduces flexibility to handle multi-subject training without introducing unneeded complexity in single-subject or homogeneous datasets.

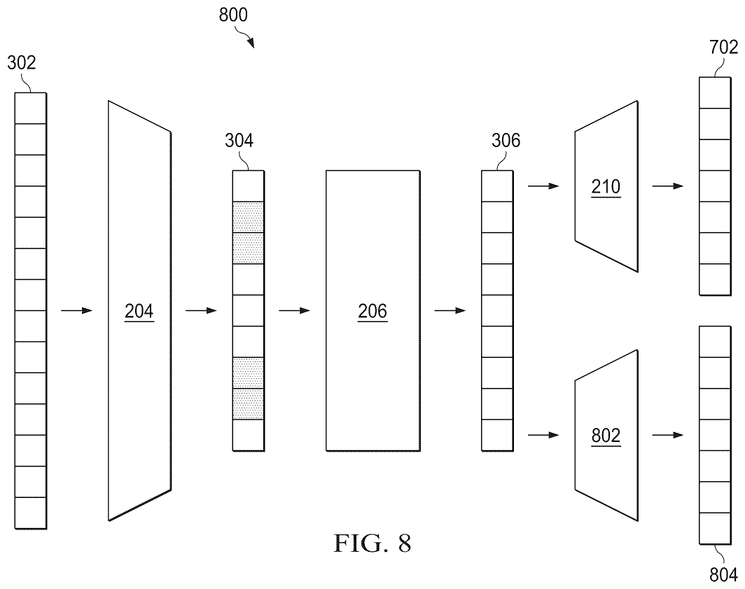

FIG. 8 is a block diagram 800 illustrating example data flow between the encoder 204, the transformer 206, and the pretraining heads 208 during the training process, according to some examples. In the example of FIG. 8, the pretraining heads 208 include the decoder 210 and a second decoder 802 to facilitate pretraining on a multimodal dataset. In the example of FIG. 8, the input signal 302 may represent sensor data of multiple modalities—such as, for example, EEG data and EOG data—concatenated end-to-end. Accordingly, the masked encoded representation 304 and the contextual features 306 generated by the feature extractor 202 may contain temporally aligned information capturing multiple distinct sensor modalities.

To accommodate this multimodal input, the pretraining heads 208 may include multiple decoders specialized for reconstructing different target modalities from the shared contextual features 306. In some examples, each decoder may process the contextual features 306 sequentially, while in other examples, the decoders may operate in parallel on shared or partitioned portions of the contextual features 306. For instance, the decoder 210 may reconstruct a predicted frequency-domain representation corresponding to the first modality (e.g., EEG data), while the second decoder 802 may reconstruct a predicted frequency-domain representation corresponding to the second modality (e.g., EOG data).

The second decoder 802 may be implemented using architectural principles similar to the decoder 210. For example, the second decoder 802 may include one or more neural network layers, such as fully connected (dense) layers, convolutional layers, transposed convolution (deconvolution) layers, or a combination thereof. In some implementations, the second decoder 802 mirrors the structure of the decoder 210, applying sequences of linear transformations and non-linear activations to progressively transform the contextual features 306 into a modality-specific output. Additionally, the second decoder 802 may include normalization layers (such as batch normalization), dropout layers for regularization, and residual connections to stabilize training. Depending on the task, the second decoder 802 may upsample or interpolate the contextual features to match the temporal and spectral resolution needed for reconstructing the reference frequency-domain representation of the second modality. Although the decoders 210 and 802 share architectural similarities, each decoder may learn separate parameters specialized for reconstructing the corresponding modality.

During training, the training application 118 may compute a reconstruction loss for each decoder. For example, the decoder 210 may output a predicted frequency-domain representation 702 corresponding to the first modality, and the second decoder 802 may output a predicted frequency-domain representation 804 corresponding to the second modality. Each predicted frequency-domain representation may be compared against a corresponding reference frequency-domain representation generated by applying a time-frequency decomposition, such as a STFT, to the appropriate portion of the original input signal 302. In various implementations, the predicted and reference frequency-domain representations are normalized by z-scoring along the time axis, emphasizing relative spectral variations over absolute amplitudes.

The reconstruction losses for each modality may be computed using one or more suitable loss functions, including MSE, MAE, Huber loss, KL divergence, cosine similarity loss, or weighted combinations thereof. For example, a total loss [symbol] ₜₒₜₐₗ may be computed as a weighted sum of the individual modality-specific losses, for example, according to Equation (4):

$${ℒ}_{total} = {λ}_{1} {ℒ}_{modality 1} + {λ}_{2} {ℒ}_{modality 2} \qquad ( 4 )$$

In Equation (4), λ₁ and λ₂ are weighting factors that may be tuned according to training objectives. The training application 118 may adjust parameters of the encoder 204, transformer 206, and/or the decoders 210 and 802 based on the computed total loss. Suitable optimization algorithms—such as Adam, AdamW, or Lookahead—may be used to apply gradient-based updates, with optional dynamic learning rate schedules (such as cosine annealing or step decay) to improve convergence stability.

Training the machine learning model with multiple decoders across multimodal datasets provides several technical advantages. By exposing the encoder 204 and transformer 206 to diverse but structurally related frequency-domain signals during pretraining, the model is encouraged to learn generalizable spectral representations that transcend individual modalities. For example, while EEG and EOG signals reflect different physiological processes, both share common time-frequency characteristics such as rhythmic oscillations, transient bursts, and spectral transitions. Jointly training on multiple modalities thus regularizes the feature extractor 202, reducing the risk of overfitting to modality-specific artifacts and encouraging the discovery of deeper, modality-agnostic structures. As a result, the pretrained model may exhibit improved robustness to domain shifts, enhanced transferability to new modalities or recording setups, and stronger generalization across subject populations. Furthermore, multimodal training may increase the effective size and diversity of the pretraining corpus, accelerating convergence and improving downstream performance even in low-data or cross-modal scenarios.

Returning to FIG. 5, in the example message sequence chart 500, after training the feature extractor 202, the training application 118 fine-tunes the pretrained feature extractor 202 (at operation 510), for example, for specific inference modalities and/or tasks. In various implementations, the training application 118 removes the pretraining-specific head (e.g., the decoder 210) and attaches a task-specific inference head 212 (such as, for example, a linear classification or regression layer). The training application 118 may provide a set of labeled, domain- and/or task-specific training data to the feature extractor 202 with the inference head 212 attached, and fine-tune parameters of the feature extractor 202 and/or the inference head 212 to minimize a supervised loss defined over downstream task labels.

To ensure compatibility between the pretraining and fine-tuning data, the training application 118 may resample the fine-tuning dataset to match the sampling frequency of the dataset used during pretraining. This alignment preserves the frequency selectivity of the convolutional encoder 204 and maintains the temporal consistency of the contextual relationships learned by the transformer 206.

When the resampled fine-tuning signal is shorter than the pretraining input length, the training application 118 may apply zero-padding. When the signal is longer, the training application 118 may segment it into overlapping windows of fixed length corresponding to the pretraining configuration. The feature extractor 202 may process each window independently, and the resulting contextual feature representations may be averaged to produce a temporally consistent embedding suitable for downstream learning or inference.

In some implementations, to promote channel-independence, the training application 118 restructures multi-channel input data by concatenating channels along the batch dimension and processes them as single-channel instances during both pretraining and fine-tuning. This design supports modular feature extraction and simplifies cross-modal adaptation.

This fine-tuning strategy allows the pretrained model to retain its learned frequency- and time-domain representations while adapting efficiently to new signal types, tasks, and domains with limited labeled data. As a result, the system may improve generalization across modalities and reduces the computational burden of retraining large portions of the model during domain transfer.

In the example message sequence chart 500, the training application 118 deploys the trained feature extractor 202 to the inference platform 108 (at operation 512), for example, according to any of the previously described techniques. In various implementations, the training application 118 trains and or deploys task-specific inference heads 212 to the inference platform, for example, according to any of the previously described techniques.

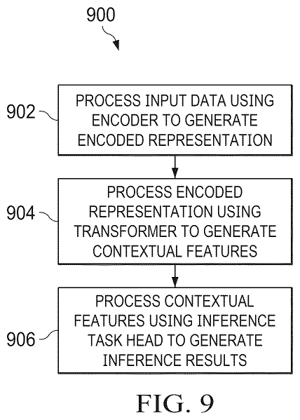

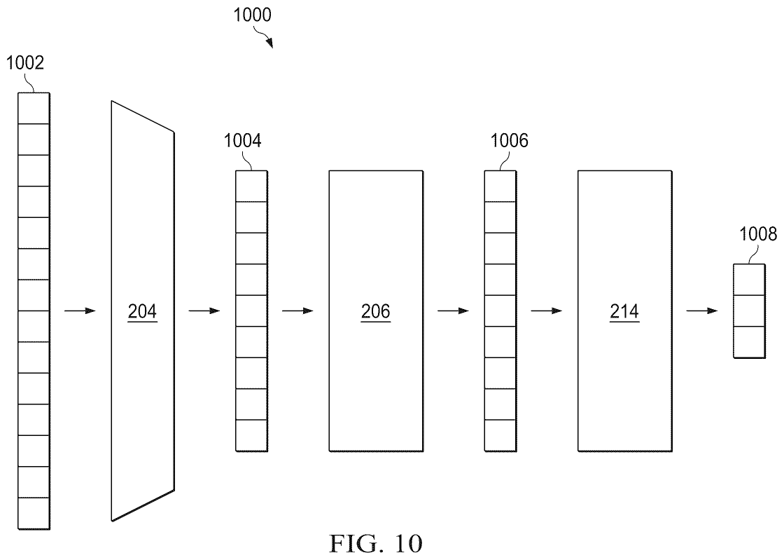

In the example message sequence chart 500, the inference application 128 performs inference using the trained machine learning models deployed to the model store 130 (at operation 514). FIG. 9 is a flowchart illustrating an example process 900 for performing inference using the trained feature extractor 202, according to some examples. FIG. 10 is a block diagram 1000 illustrating example data flow between the encoder 204, the transformer 206, and the inference head 214 during the inference process, according to some examples. Referring collectively to FIGS. 9 and 10, in the example process 900, the inference application 128 provides input data—such as the input signal 1002—to the trained encoder 204 (at block 902).

In various implementations, the input signal 1002 represents features from a sensor modality different from the modality of the training data (e.g., the input signal 302). In some examples, the input signal 1002 and the input signal 302 correspond to the same modality. In various implementations, the input signal 1002 is structured and dimensioned similarly to the input signal 302, for example, following any of the previously described preprocessing techniques. In some examples, the inference application 128 resamples the input signal 1002 to match the sampling rate of the input signal 302, facilitating compatibility between the pretrained encoder 204 and the frequency content of the new input.

In response to the resampled input signal 1002 being shorter than the input length expected by the feature extractor 202, the inference application 128 may apply zero-padding to extend the input signal 1002 to the expected dimensionality. In response to the resampled input signal 1002 being longer than the expected input length, the inference application 128 may split the input signal 1002 into overlapping windows, pass each window independently through the feature extractor 202, and aggregate the outputs across windows. In some examples, aggregation may involve simple averaging, while in other examples, outputs may be weighted or smoothed based on the relative temporal positioning of each window, facilitating continuous and stable output predictions over time.

The proper alignment of the sampling rates between the input signal 1002 and the original training signal 302 facilitates several technical benefits. Frequency-domain features—such as oscillatory components, rhythmic bursts, and harmonic structures—may be sampling-rate dependent. When sampling rates are misaligned, the spectral bins produced by the encoder 204 and transformer 206 during pretraining would no longer match the spectral distribution of the new input, degrading inference accuracy. Resampling the input signal 1002 to match the original training conditions preserves the correspondence between frequency features and model expectations, facilitating consistent and accurate feature extraction at inference time.

Following any resampling and windowing operations, the inference application 128 provides the processed input signal 1002 to the encoder 204. The trained encoder 204 processes the input signal 1002 to generate an encoded representation 1004. The encoded representation 1004 may have the same structural format as the encoded representation 304 generated during pretraining—for example, a matrix where each column corresponds to a learned feature vector representing a localized receptive field segment of the input signal 1002.

In the example process 900, the inference application 128 then passes the encoded representation 1004 to the transformer 206 (at block 904). The transformer 206 may process the encoded representation 1004 to generate contextual features 1006. In various implementations, the transformer 206 applies positional encodings and self-attention operations across the encoded patches to model long-range temporal dependencies, facilitating inference tasks that rely on global patterns in the input data. The contextual features 1006 output by the transformer 206 may thus represent temporally-enriched, globally-aware feature embeddings that incorporate information aggregated across multiple patches of the input signal 1002.

In the example process 900, the inference application 128 passes the contextual features 1006 to one or more inference heads 212, such as the decoder head 214 (at block 906). The inference head 214 processes the contextual features 1006 to generate inference results 1008. The inference results 1008 may correspond to task-specific outputs—for example, class labels, regression values, segmentation maps, or anomaly scores—depending on the configuration of the selected inference head. In some implementations, different inference heads may be selected dynamically based on the modality of the input signal 1002 or the specific downstream application needs.

In various implementations, the inference application 128 may use multiple inference heads in parallel to produce multiple outputs from the same contextual features 1006. For example, one inference head may predict the physiological state associated with the input signal 1002, while another inference head predicts the quality or reliability of the input signal 1002. This modular inference framework facilitates flexible, application-specific use of the pretrained feature extractor 202 and supports broad generalization across heterogeneous datasets and tasks.

In various implementations, the inference application 128 dynamically handles cases where only a subset of modalities are available at inference time. In response to only a partial set of modalities being available, the inference application 128 selectively applies the feature extractor 202 and the appropriate inference heads to the available input data, facilitating robust operation even under missing data conditions.

By combining flexible input resampling, zero-padding, windowed aggregation, dynamic inference head selection, missing modality handling, and calibration-free deployment, the inference system 100 facilitates robust, reliable, and generalizable application of pretrained machine learning models across a wide variety of physiological signal types, device configurations, and real-world use cases.

### Additional Enumerated Examples

The following paragraphs provide examples of systems, methods, and devices implemented in accordance with this specification.

Example 1. A computer-implemented method, comprising: processing a time-series input signal using an encoder to produce an encoded representation; segmenting the encoded representation into a plurality of patches; applying a masking operation to a subset of the patches to produce a masked encoded representation; processing the masked encoded representation using a transformer to generate contextual features; processing the contextual features using a decoder to generate a predicted frequency-domain representation of the time-series input signal; and adjusting parameters of the encoder and parameters of the transformer to minimize a loss between the predicted frequency-domain representation and a reference frequency-domain representation derived from the time-series input signal.

Example 2. The method of example 1, wherein the masking operation includes masking one or more consecutive sequences of fixed-sized patches from the encoded representation.

Example 3. The method of example 1, wherein the time-series input signal includes a first modality and a second modality, the method further comprising: processing the contextual features using a first decoder to generate a first predicted frequency-domain representation of the time-series input signal; processing the contextual features using a second decoder to generate a second predicted frequency-domain representation of the time-series input signal; and adjusting parameters of the encoder and parameters of the transformer to minimize losses between (i) the first predicted frequency-domain representation and a first reference frequency-domain representation derived from the first modality and (ii) the second predicted frequency-domain representation and a second reference frequency-domain representation derived from the second modality.

Example 4. The method of example 1, further comprising: fine tuning the encoder and transformer on labeled fine-tuning data; providing the fine-tuned encoder and transformer for inference on input data; wherein the input data corresponds to a modality different from a modality of the time-series input signal.

Example 5. The method of example 4, further comprising resampling the input data to match a sampling rate of the time-series input signal.

Example 6. The method of example 5, further comprising zero-padding resampled input data having a temporal length shorter than a temporal length of the time-series input signal.

Example 7. The method of example 5, further comprising: dividing the resampled input data having a temporal length longer than a temporal length of the time-series input signal into a plurality of overlapping windows; processing each window using the encoder and the transformer to generate corresponding inference contextual features; and averaging the inference contextual features to generate an aggregated representation.

Example 8. The method of example 1, wherein the encoded representation includes a subject-specific embedding, the method further comprising: adjusting the subject-specific embedding to minimize the loss between the predicted frequency-domain representation and the reference frequency-domain representation derived from the time-series input signal.

Example 9. The method of example 1, wherein: the encoder includes a convolutional neural network configured to extract local temporal features from the time-series input signal to generate the encoded representation; and the transformer includes a temporal self-attention model configured to extract global temporal features from the encoded representation to generate the contextual features.

Example 10. A non-transitory computer-readable medium comprising executable instructions that, when executed by an electronic processor, causes the electronic processor to perform the method of example 1.

Example 11. A computer-implemented method, comprising: processing input data using an encoder to generate an encoded representation; processing the encoded representation using a transformer to generate contextual features; and processing the contextual features using an inference task head to generate inference results; wherein the encoder and the transformer are pretrained using a time-series input signal by: applying a masking operation to a subset of patches of a training encoded representation of the time-series input signal generated by the encoder, processing the masked encoded representation using the transformer to generate training contextual features, processing the training contextual features using a decoder to generate a predicted frequency-domain representation of the time-series input signal, and adjusting parameters of the encoder and parameters of the transformer to minimize a loss between the predicted frequency-domain representation and a reference frequency-domain representation derived from the time-series input signal.

Example 12. The method of example 11, wherein the masking operation includes masking one or more consecutive sequences of fixed-sized patches from the training encoded representation.

Example 13. The method of example 11, wherein the time-series input signal includes a first modality and a second modality, and the encoder and the transformer are pretrained by: processing the training contextual features using a first decoder to generate a first predicted frequency-domain representation of the time-series input signal; processing the training contextual features using a second decoder to generate a second predicted frequency-domain representation of the time-series input signal; and adjusting parameters of the encoder and parameters of the transformer to minimize losses between (i) the first predicted frequency-domain representation and a first frequency-domain representation derived from the first modality and (ii) the second predicted frequency-domain representation and a second reference frequency-domain representation derived from the second modality.

Example 14. The method of example 11, wherein the input data corresponds to a modality different from a modality of the time-series input signal.

Example 15. The method of example 11, further comprising resampling the input data to match a sampling rate of the time-series input data.

Example 16. The method of example 15, further comprising zero-padding resampled input data having a temporal length shorter than a temporal length of the time-series input signal.

Example 17. The method of example 15, further comprising: dividing the resampled input data having a temporal length longer than a temporal length of the time-series input signal into a plurality of overlapping windows; processing each window using the encoder and the transformer to generate corresponding contextual representations; and averaging the contextual representations to generate the contextual features.

Example 18. The method of example 11, wherein the encoded representation includes a subject-specific embedding, the subject-specific embedding learned during pretraining to minimize the loss between the predicted frequency-domain representation and the reference frequency-domain representation.

Example 19. The method of example 11, wherein: the encoder includes a convolutional neural network configured to extract local temporal features from the time-series input signal to generate the encoded representation; and the transformer includes a temporal self-attention model configured to extract global temporal features from the encoded representation to generate the contextual features.

Example 20. A system comprising: non-transitory computer-readable storage media storing instructions; and an electronic processor configured to execute the instructions, wherein executing the instructions causes the electronic processor to perform the method of example 11.

The foregoing description is merely illustrative in nature and does not limit the scope of the disclosure or its applications. The broad teachings of the disclosure may be implemented in many different ways. While the disclosure includes some particular examples, other modifications will become apparent upon a study of the drawings, the text of this specification, and the following claims. In the written description and the claims, one or more processes within any given method may be executed in a different order or processes may be executed concurrently or in combination with each other without altering the principles of this disclosure. Similarly, instructions stored in a non-transitory computer-readable medium may be executed in a different order or concurrently without altering the principles of this disclosure. Unless otherwise indicated, the numbering or other labeling of instructions or method steps is done for convenient reference and does not necessarily indicate a fixed sequencing or ordering.

It should also be noted that a plurality of hardware and software-based devices, as well as a plurality of different structural components may be utilized in various implementations. Aspects, features, and instances may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one instance, the electronic based aspects of the invention may be implemented in software (for example, stored on non-transitory computer-readable medium) executable by one or more processors. As a consequence, it should be noted that a plurality of hardware and software-based devices, as well as a plurality of different structural components may be utilized to implement the invention. For example, “control units” and “controllers” described in the specification can include one or more electronic processors, one or more memories including a non-transitory computer-readable medium, one or more input/output interfaces, and various connections (for example, a system bus) connecting the components.

Unless the context of their usage unambiguously indicates otherwise, the articles “a,” “an,” and “the” should not be interpreted to mean “only one.” Rather, these articles should be interpreted to mean “at least one” or “one or more.” Likewise, when the terms “the” or “said” are used to refer to a noun previously introduced by the indefinite article “a” or “an,” the terms “the” or “said” should similarly be interpreted to mean “at least one” or “one or more” unless the context of their usage unambiguously indicates otherwise.

It should also be understood that although certain drawings illustrate hardware and software located within particular devices, these depictions are for illustrative purposes only. In some embodiments, the illustrated components may be combined or divided into separate software, firmware, and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable connections or links.

Thus, in the claims, if an apparatus or system is claimed, for example, as including an electronic processor or other element configured in a certain manner, for example, to make multiple determinations, the claim or claim element should be interpreted as meaning one or more electronic processors (or other element) where any one of the one or more electronic processors (or other element) is configured as claimed, for example, to make some or all of the multiple determinations collectively. To reiterate, those electronic processors and processing may be distributed.

Spatial and functional relationships between elements—such as modules—are described using terms such as (but not limited to) “connected,” “engaged,” “interfaced,” and/or “coupled.” Unless explicitly described as being “direct,” relationships between elements may be direct or include intervening elements. The phrase “at least one of A, B, and C” should be construed to indicate a logical relationship (A OR B OR C), where OR is a non-exclusive logical OR, and should not be construed to mean “at least one of A, at least one of B, and at least one of C.” The term “set” does not necessarily exclude the empty set. For example, the term “set” may have zero elements. The term “subset” does not necessarily require a proper subset. For example, a “subset” of set A may be coextensive with set A, or include elements of set A. Furthermore, the term “subset” does not necessarily exclude the empty set.

In the figures, the directions of arrows generally demonstrate the flow of information—such as data or instructions. The direction of an arrow does not imply that information is not being transmitted in the reverse direction. For example, when information is sent from a first element to a second element, the arrow may point from the first element to the second element. However, the second element may send requests for data to the first element, and/or acknowledgements of receipt of information to the first element. Furthermore, while the figures illustrate a number of components and/or steps, any one or more of the components and/or steps may be omitted or duplicated, as suitable for the application and setting.

Additionally, operations (such as processes, decisions, inputs, outputs, actions, messages, interactions, events, and/or any other operations) shown in the flowcharts and/or message sequence charts may be illustrated once each and in a particular order in the drawings. However, in various implementations, the operations may be reordered and/or repeated as may be suitable. In some examples, different operations may be performed in parallel, as may be appropriate.

The term computer-readable medium does not encompass transitory electrical or electromagnetic signals or electromagnetic signals propagating through a medium—such as on an electromagnetic carrier wave. The term “computer-readable medium” is considered tangible and non-transitory. The functional blocks, flowchart elements, and message sequence charts described above serve as software specifications that can be translated into computer programs by the routine work of a skilled technician or programmer.

What is claimed is:

1. A computer-implemented method, comprising:
processing a time-series input signal using an encoder to produce an encoded representation;
segmenting the encoded representation into a plurality of patches;
applying a masking operation to a subset of the patches to produce a masked encoded representation;
processing the masked encoded representation using a transformer to generate contextual features;
processing the contextual features using a decoder to generate a predicted frequency-domain representation of the time-series input signal; and
adjusting parameters of the encoder and parameters of the transformer to minimize a loss between the predicted frequency-domain representation and a reference frequency-domain representation derived from the time-series input signal.

2. The method of claim 1, wherein the masking operation includes masking one or more consecutive sequences of fixed-sized patches from the encoded representation.

3. The method of claim 1, wherein the time-series input signal includes a first modality and a second modality, the method further comprising:
processing the contextual features using a first decoder to generate a first predicted frequency-domain representation of the time-series input signal;
processing the contextual features using a second decoder to generate a second predicted frequency-domain representation of the time-series input signal; and
adjusting parameters of the encoder and parameters of the transformer to minimize losses between (i) the first predicted frequency-domain representation and a first reference frequency-domain representation derived from the first modality and (ii) the second predicted frequency-domain representation and a second reference frequency-domain representation derived from the second modality.

4. The method of claim 1, further comprising:
fine tuning the encoder and transformer on labeled fine-tuning data;
providing the fine tuned encoder and transformer for inference on input data;
wherein the input data corresponds to a modality different from a modality of the time-series input signal.

5. The method of claim 4, further comprising resampling the input data to match a sampling rate of the time-series input signal.

6. The method of claim 5, further comprising zero-padding resampled input data having a temporal length shorter than a temporal length of the time-series input signal.

7. The method of claim 5, further comprising:
dividing the resampled input data having a temporal length longer than a temporal length of the time-series input signal into a plurality of overlapping windows;
processing each window using the encoder and the transformer to generate corresponding inference contextual features; and
averaging the inference contextual features to generate an aggregated representation.

8. The method of claim 1, wherein the encoded representation includes a subject-specific embedding, the method further comprising:
adjusting the subject-specific embedding to minimize the loss between the predicted frequency-domain representation and the reference frequency-domain representation derived from the time-series input signal.

9. The method of claim 1, wherein:
the encoder includes a convolutional neural network configured to extract local temporal features from the time-series input signal to generate the encoded representation; and
the transformer includes a temporal self-attention model configured to extract global temporal features from the encoded representation to generate the contextual features.

10. A non-transitory computer-readable medium comprising executable instructions that, when executed by an electronic processor, causes the electronic processor to perform the method of claim 1.

11. A computer-implemented method, comprising:
processing input data using an encoder to generate an encoded representation;
processing the encoded representation using a transformer to generate contextual features; and
processing the contextual features using an inference task head to generate inference results;
wherein the encoder and the transformer are pretrained using a time-series input signal by:
applying a masking operation to a subset of patches of a training encoded representation of the time-series input signal generated by the encoder,
processing the masked encoded representation using the transformer to generate training contextual features,
processing the training contextual features using a decoder to generate a predicted frequency-domain representation of the time-series input signal, and
adjusting parameters of the encoder and parameters of the transformer to minimize a loss between the predicted frequency-domain representation and a reference frequency-domain representation derived from the time-series input signal.

12. The method of claim 11, wherein the masking operation includes masking one or more consecutive sequences of fixed-sized patches from the training encoded representation.

13. The method of claim 11, wherein the time-series input signal includes a first modality and a second modality, and the encoder and the transformer are pretrained by:
processing the training contextual features using a first decoder to generate a first predicted frequency-domain representation of the time-series input signal;
processing the training contextual features using a second decoder to generate a second predicted frequency-domain representation of the time-series input signal; and
adjusting parameters of the encoder and parameters of the transformer to minimize losses between (i) the first predicted frequency-domain representation and a first frequency-domain representation derived from the first modality and (ii) the second predicted frequency-domain representation and a second reference frequency-domain representation derived from the second modality.

14. The method of claim 11, wherein the input data corresponds to a modality different from a modality of the time-series input signal.

15. The method of claim 11, further comprising resampling the input data to match a sampling rate of the time-series input data.

16. The method of claim 15, further comprising zero-padding resampled input data having a temporal length shorter than a temporal length of the time-series input signal.

17. The method of claim 15, further comprising:
dividing the resampled input data having a temporal length longer than a temporal length of the time-series input signal into a plurality of overlapping windows;
processing each window using the encoder and the transformer to generate corresponding contextual representations; and
averaging the contextual representations to generate the contextual features.

18. The method of claim 11, wherein the encoded representation includes a subject-specific embedding, the subject-specific embedding learned during pretraining to minimize the loss between the predicted frequency-domain representation and the reference frequency-domain representation.

19. The method of claim 11, wherein:
the encoder includes a convolutional neural network configured to extract local temporal features from the time-series input signal to generate the encoded representation; and
the transformer includes a temporal self-attention model configured to extract global temporal features from the encoded representation to generate the contextual features.

20. A system comprising:
non-transitory computer-readable storage media storing instructions; and
an electronic processor configured to execute the instructions, wherein executing the instructions causes the electronic processor to perform the method of claim 11.
